(19) United States
(12) Patent Application Publication
(10) Pub. No.: US 2017/0182196 A1
(43) Pub. Date: Jun. 29, 2017

(54) APPARATUS CONFIGURED FOR COLLECTION AND STERILIZATION OF EXPECTORATES

(71) Applicant: Nilin Patel, Raleigh, NC (US)

(72) Inventor: Nilin Patel, Raleigh, NC (US)

(21) Appl. No.: 15/461,633

(22) Filed: Mar. 17, 2017

(51) Int. Cl. A61L 2/26; A61B 10/00; A61L 2/10

(52) U.S. Cl. CPC A61L 2/26; A61L 2/10; A61B 10/0038; A61L 2202/20; A61L 2202/11; A61L 2202/122; A61L 2202/121

(57) ABSTRACT

Disclosed is an apparatus configured for collection and sterilization of expectorates. According to the embodiments of the invention, the apparatus includes a receptacle having an interior space defined by a bottom wall, and one or more side walls, an opening at a top portion for selectively receiving a separator for the expectorates to pass therethrough and be received at the interior space, and a power source, one or more light sources, such as ultraviolet (UV) light source radiating rays of sufficient wavelengths capable of sterilizing the expectorates received at the interior space, a reflective source surrounding the interior space of the receptacle for substantially reflecting the radiated rays throughout the interior space to effectively sterilize the expectorates received at the interior space, and a receptacle lid removably engaging the separator to close the opening at the top portion of the receptacle.

### FIELD OF THE INVENTION

The present application generally relates to sanitation and hygiene and in particular, it relates to a device employing one or more light sources, such as ultraviolet (UV) light sources or light sources of comparable wavelengths. More particularly, embodiments of the present invention are directed towards an apparatus that would enable collection of expectorates such as sputum, spit and the like, sterilization of the collected expectorates before disposal for preventing infections or diseases that may be resulting due to presence of pathogenic microbial agents like viruses, bacteria, fungi, and other forms of micro organisms.

### BACKGROUND

We all know about importance of sanitation and hygiene and how sanitation can transform lives of people. There may be several reasons due to which sanitation and hygiene issues may arise, such as for example, lots of people defecate in the open, without access to even basic toilets or hand washing facilities. Another important factor that results in the sanitation and hygiene issues is improper methods adapted for disposal of expectorates or contaminants (e.g spit, cough, phlegm or sputum and the like) coming out from hospitals or various health care facilities or households. Expectoration in open environment directly by people or improper disposal of the expectorates from the hospitals or various health care facilities can give rise to various infectious diseases resulting due to the presence of pathogenic microbial agents like viruses, bacteria, fungi, and other forms of micro organisms. Such diseases can even transform into critical illness and may result in deaths.

In the past, various devices or containers for collecting sputum, spit etc are devised. They may serve the purpose of collecting expectorates at one place, which can later be disposed at some safe location. However, such devices or containers fail to disinfect the contaminant or expectorates before disposal. Further, even after the disposal of the contaminants out of such containers, many a times such containers are found carrying contaminants like viruses, bacteria, fungi, and other forms of micro organisms. It is important that such containers are made contaminant free before they are used again.

Further, in the past, several devices are found to be proposing use of UV light sources to disinfect contaminants such as sputum or spit, which may be collected in those devices for later disposal, for instance the devices proposed in Chinese utility patent or application CN2140743 and CN 201500310. It is observed that such devices or systems are directed either towards emphasizing on comfort and specific aesthetic appearance of the device or cleaning process of the device after the use using some brushes configured in the device which is actuated using a motor.

The above information is presented as background information only to help the reader to understand the present invention. Applicant has made no determination and makes no assertion as to whether any of the above might be applicable as prior art with regard to the present application.

In the light of above discussed background arts, there is a need for an apparatus or device that would overcome above stated issues. The proposed invention discloses a sanitation device simple in its construction, portable and that can effectively be deployed by health care facilities or even in the household by common people.

### BRIEF SUMMARY

Before the present systems and methods, enablement are described, it is to be understood that this application is not limited to the particular systems, and methodologies described, as there can be multiple possible embodiments which are not expressly illustrated in the present disclosures. It is also to be understood that the terminology used in the description is for the purpose of describing the particular versions or embodiments only, and is not intended to limit the scope of the present application.

It is an objective of present invention to provide an apparatus that would enable collection and sterilization of expectorates in hospitals or various health care facilities or households.

It is another objective of present invention to provide an apparatus flexible to use everywhere and that may effectively sterilize or disinfect expectorates using one or more light sources, such as ultraviolet (UV) light sources or light sources of wavelengths comparable to the wavelengths of the UV light sources that may be sufficient for disinfecting or sterilization of expectorates.

Another objective of the present invention is to provide an apparatus that may be portable, and that may also be installed as stationary device in various hospitals or healthcare facilities.

Yet another objective of the present invention is to provide a simple and easy to use sterilizing device that would prevent users from various infectious diseases resulting due to the presence of pathogenic microbial agents like viruses, bacteria, fungi, and other forms of micro organisms.

According to embodiments of the present invention, there is provided an apparatus for collection and sterilization of expectorates. The apparatus includes a receptacle including an interior space defined by a bottom wall, and one or more side walls, an opening at a top portion for selectively receiving a separator for the expectorates to pass therethrough and be received at the interior space, and a power source. The power source is configured on a bottom portion of the receptacle or alternatively be configured onto the side walls and a receptacle lid. The separator includes a plurality of openings to pass expectorates to the interior space.

According to the embodiments, the apparatus further includes one or more light sources, such as ultraviolet (UV) light sources or light sources of comparable wavelengths that may be capable of sterilizing the expectorates received at the interior space, wherein the one or more light sources are powered by the power source, and a reflective source surrounding the interior space of the receptacle for substantially reflecting the radiated rays throughout the interior space to effectively sterilize the expectorates received at the interior space. The reflective source including a reflective foil. The reflective source including one or more reflective mirrors. The one or more light sources configured onto the bottom wall or the one or more side walls of the receptacle. The one or more light sources configured on an underside face of the receptacle lid, the underside face being opposite to the bottom wall of the receptacle. Further, the one or more light sources are configured externally. The rays from the externally configured one or more light sources is directed to enter the interior space of the receptacle using a directing mechanism.

According to the embodiment, the apparatus further includes a receptacle lid removably engaging the separator to close the opening at the top portion of the receptacle. The receptacle lid having a top flap hingedly configured thereon to operate in an open state, and a closed state. The top flap when operated in the open state deactivates the one or more light sources. The top flap when operated in the closed state activates the one or more light sources. The receptacle lid includes a funnel headed tube assembly configured thereon. The funnel headed tube assembly comprises of an elongated flexible tube of a predetermined length including an upper end, and a lower end, and including a cavity with length substantially equal to the predetermined length of the elongated flexible tube; a funnel body configured on the upper end of the elongated flexible tube for receiving the expectorates; and a funnel lid for closure of the funnel body when not in use. The funnel headed tube assembly directs expectorates to the interior space of the receptacle for collection and sterilization.

According to the embodiment, the apparatus further includes an insert that can be removably disposed within the interior space of the receptacle.

In addition to the above aspects, embodiments and advantages described in this summary, further aspects and advantages will become apparent with reference to the drawings and the detailed description that follows.

### BRIEF DESCRIPTION OF THE DRAWINGS

The foregoing summary, as well as the following detailed description of preferred embodiments, is better understood when read in conjunction with the appended drawings. There is shown in the drawings example embodiments, however, the application is not limited to the specific system and method disclosed in the drawings.

FIG. 1 illustrates an exploded view of an apparatus configured for collection and sterilization of expectorates, in accordance with an embodiment of the present invention.

FIG. 2A-2C illustrates inside view of a receptacle of the apparatus shown in FIG.1 with possible various configurations of one or more light sources, such as ultraviolet (UV) light source or light sources of comparable wavelengths, according to the embodiment of the present invention.

FIG. 3 illustrates an assembled inside view of the apparatus of FIG. 1 with a top flap of a receptacle lid shown in an opened state for receiving expectorates, according to the embodiment of the present invention.

FIG. 4 illustrates an alternative exemplary embodiment of the apparatus configured for collection and sterilization of expectorates.

FIG. 5 illustrates another alternative exemplary embodiment of the apparatus configured for collection and sterilization of expectorates.

FIG. 6 illustrates yet another alternative exemplary embodiment of the apparatus configured for collection and sterilization of expectorates.

FIG. 7 illustrates use of the apparatus shown in FIG. 6 in hospitals or health care facilities for the convenience of patients, in accordance with an exemplary embodiment.

### DETAILED DESCRIPTION

Some embodiments, illustrating its features, will now be discussed in detail. The words “comprising”, “having”, “containing” and “including”, “comprises of” and other forms thereof, are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise. Further, in the context of the present invention the terms ‘disinfect’, ‘sterilize’ and other forms thereof are synonymously used throughout the description. Further, in the context of the present invention the terms ‘contaminants’, ‘expectorates’ and other forms thereof are synonymously used throughout the description to denote pathogenic microbial agents like viruses, bacteria, fungi, and other forms of micro organisms.

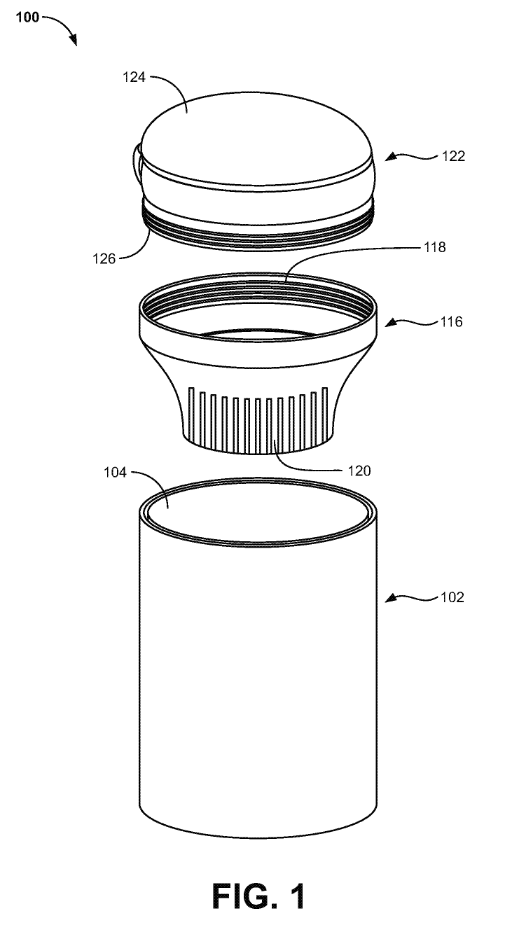

Further, the following detailed description is intended to provide example implementations to one of ordinary skill in the art, and is not intended to limit the invention, as one of ordinary skill in the art will understand that variations can be substituted that are within the scope of the invention as described. Reference will now be made to the accompanying drawings: FIGS. 1-7.

Referring to FIG. 1, there is shown an exploded view of an apparatus 100 configured for collection and sterilization of expectorates, in accordance with an embodiment of the present invention. As shown, the apparatus includes a receptacle 102 with an opening 104 at a top portion of the receptacle 102 to receive a separator 116 for expectorates to pass therethrough and be received at the receptacle 102. According to the embodiment, the separator 116 may include a plurality of openings or passages 120 that would allow steady fall of expectorates from the separator 116 into the receptacle 102. The separator 116, according to the embodiment may be internally threaded 118 for facilitating engagement of the separator 116 with a receptacle lid 122. The receptacle lid 122 may include a top flap 124. The top flap 124, according to the embodiment may be configured to operate in an open state, and a closed state. The top flap 124 may be hingedly coupled to the receptacle lid 122 to operate in the open state and the closed state. However, it should be understood any other mechanical equivalent techniques may be used to implement the same that would allow the top flap 124 to operate in the open state and the closed state. The receptacle lid 122 may also be suitably threaded 126 at its bottom to facilitate mechanical engagement of the receptacle lid 122 with the threads 118 present in the interior region of the separator 116.

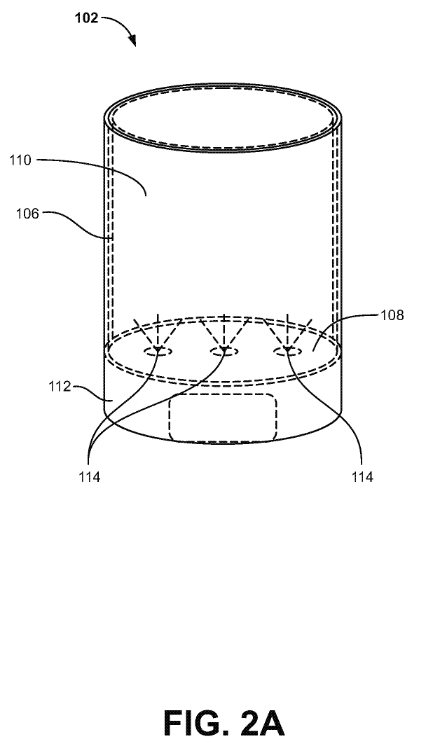

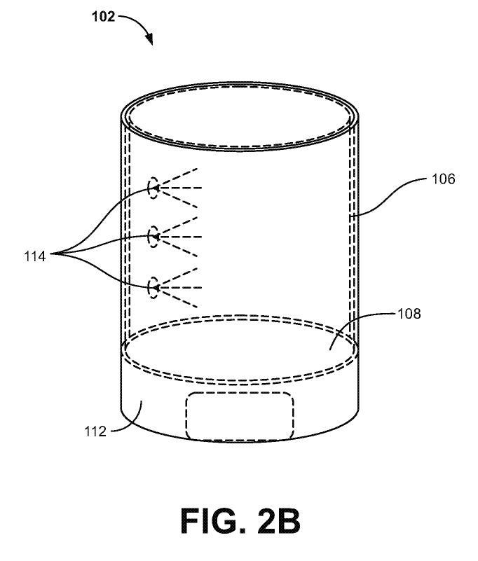

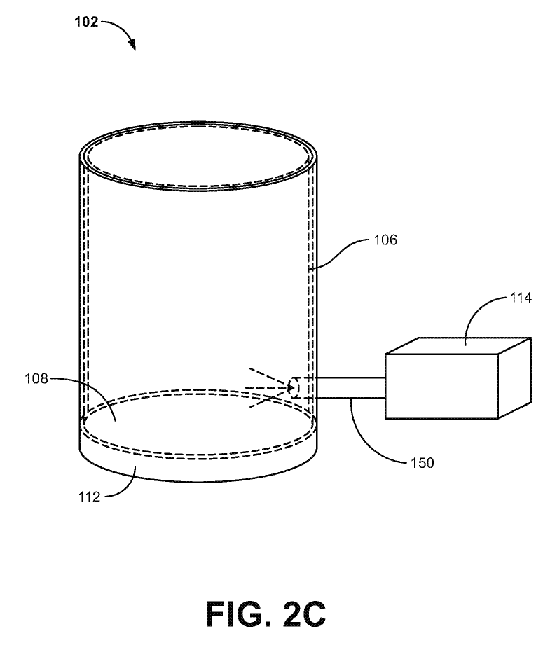

Referring to FIGS. 2A-2C which illustrates inside view of the receptacle 102 of the apparatus shown in FIG.1 with possible configurations of one or more light sources, such as ultraviolet (UV) source or light sources of comparable wavelengths, according to the embodiment of the present invention. As shown, the receptacle 102 includes an interior space defined by a bottom wall 108, and one or more side walls 110, an opening 104 at the top portion for receiving the separator 116. Besides this, the receptacle 102 may preferably include one or more ultraviolet (UV) light sources 114 for radiating rays of sufficient wavelengths capable of sterilizing expectorate received at the interior space. According to some other embodiment, the light sources of comparable wavelength with respect to UV sources may also be employed that can radiate rays of sufficient energy capable of sterilizing expectorate received at the interior space. For example, the rays of sufficient wavelength preferably include germicidal wavelengths of 185-254 nanometers (nm) which is a part of ultraviolet light spectrum and posses a high frequency sufficient for killing bacteria, viruses, and other pathogens.

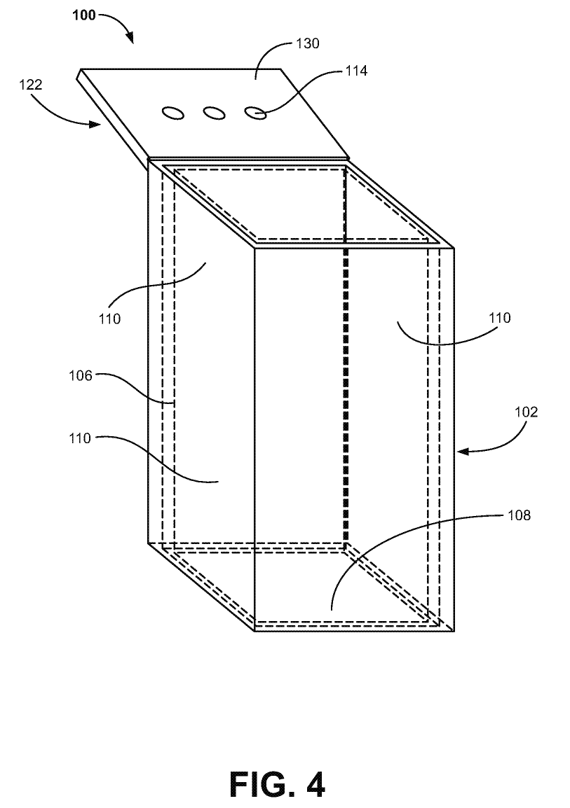

According to the preferred embodiment, the one or more ultraviolet (UV) light sources 114 or the light sources of comparable wavelengths may be configured at the bottom wall 108 of the receptacle 102, as shown in FIG.2A. However, according to another embodiment, it may be possible to configure the ultraviolet (UV) light sources 114 on the one or more side walls 110 as shown in FIG. 2B, it should be understood that the apparatus 100 is illustrated to be of cylindrical shape, it will essentially have one common side wall. According to another embodiment, the ultraviolet (UV) light sources 114 may also be configured onto the underside of the receptacle lid 122(as shown in FIG. 4).According to yet another embodiment, the ultraviolet (UV) light sources 114 may be externally configured outside the receptacle 102, as shown in FIG. 2C.

The ultraviolet (UV) light sources 114 when configured to be present externally, a directing mechanism 150 may be used for directing the rays radiated by the ultraviolet (UV) light sources 114 into the receptacle 102. The directing mechanism 150may include various components including but not limited to a tube connecting the externally present ultraviolet (UV) light sources 114 with the receptacle 102, such that the rays from those ultraviolet (UV) light sources 114 may be directed into the interior space of the receptacle 102. Further, as shown in FIGS. 2A-2C, the receptacle 102 includes a power source 112 preferably configure d at a bottom portion of the receptacle 102 or the side walls 110 or onto the receptacle lid 122. The power source 112 may include one or more DC power source such as batteries preferably rechargeable batteries. The apparatus 100 may optionally be configured to operate using AC power, for which apparatus 100 may be designed to support AC power supply. According to the embodiment, the ultraviolet (UV) light sources 114 may be configured to be able to automatically be switched to off or on states, when the top flap 124 is opened and closed, respectively for a predefined time interval. As should be understood by those skilled in the art, electronic circuitry with controllers or like electronic components interfacing the ultraviolet (UV) light sources 114 and the power source 112 may be custom programmed to embody such automatic action of auto switching on and off of the ultraviolet (UV) light sources 114.

FIGS. 2A-2C further shows a reflective source 106 surrounding the interior space of the receptacle 102. As seen and represented in dotted form, the reflective source 106 surrounds the interior space such as to reflect the radiated rays by the UV sources 114 throughout the interior space. The repetitive reflective action by the reflective source 106 on the rays radiated by the UV sources may help in effectively sterilizing expectorates received at the interior space. The reflective source 106, for example may be a reflective foil that can easily be configured inside the receptacle 102 cylindrical in shape, as illustrated in FIGS. 2A-2C. According to an alternative embodiment, one or more mirrors 106 essentially having smooth surface and high reflective index can be used for the similar purpose. An exemplary embodiment shown in FIG. 4, shows use of one or more mirrors 106 as reflective source, which will be further described below.

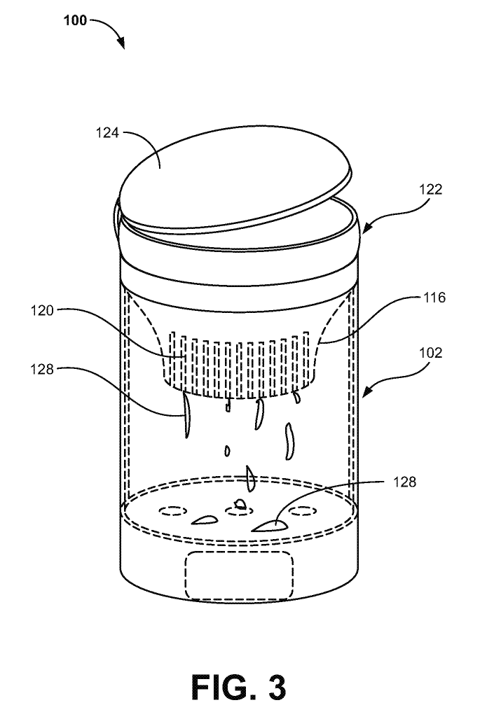

Referring to FIG. 3 shows an assembled inside view of the apparatus 100 of FIG. 1 with the top flap of the receptacle lid in opened state for receiving expectorates, according to the embodiment of the present invention. Particularly, FIG. 3 shows the apparatus of FIG. 1 in operation. In operation, when the top flap 124 is opened by a user by simply lifting the flap 124 in upward direction for expectoration, the ultraviolet (UV) light sources 114 switches off (as shown in FIG. 3) for a predefined time interval preferably but not limited to 1 second. Once the user expectorate into the apparatus 100, specifically the separator 116, expectorates 128 steadily passes through a plurality of openings 120 on the separator 116 and fall inside the interior space of the receptacle 102. As soon as the top flap 124 of the receptacle lid 122 is closed by the user, the ultraviolet (UV) light sources 114 switches on (as shown in FIGS. 2A-2C) for a predefined time interval preferably but not limited to 1 second to sterilize or disinfect expectorates 128 received or collected in the interior space of the receptacle 102. When the ultraviolet (UV) light sources 114 radiates UV rays (not shown) for the sterilization, the radiated rays strike at the reflective source 106 configured around the side wall 110 and undergo reflection and this reflection could be repetitive. When the reflected rays fall over the collected expectorates 128 preferably present on the bottom wall 108, the rays disinfect or kill the pathogenic microbial agents like viruses, bacteria, fungi, and other forms of micro organisms that may be present in expectorates. Once expectorates are disinfected or become contaminant free, the disinfected expectorates can be safely disposed without any risk of contamination of environment or infectious diseases originating from them.

Referring to FIG. 4 shows an alternative exemplary embodiment of the apparatus configured for collection and sterilization of expectorates. As shown, the apparatus 100 embodies a square shape. Unlike the embodiment described above with reference to FIGS. 1-3, the interior space of the receptacle 102is formed by four side walls 110 and a bottom wall 108.The ultraviolet (UV) light sources 114 are preferably configured on an underside face 130 of the receptacle lid 122. The underside face 130 is opposite to the bottom wall 108of the receptacle 102. The embodiment shows use of one or more mirrors 106 as reflective source configured onto the side walls 110, and the bottom wall 108.The embodiment, as shown, embodies the receptacle lid 122 configured in the form of flap 124, instead of having the flap 124 separately configured on the receptacle lid 122 as a simplified design. Further, since the ultraviolet (UV) light sources 114 are preferably configured on an underside face 130 of the receptacle lid 122, the use of the separator 116 may be avoided, and the apparatus 100 may be designed such that the receptacle lid 122may be hingedly configured on the top portion of the receptacle 102 for closing the opening 104 of the receptacle 102.

Further referring to FIG.4, operative procedure is likely the same as described above with reference to FIG.3. When the receptacle lid 122 is opened by the user by simply lifting the lid 122 in upward direction for expectoration, the ultraviolet (UV) light sources 114 switches off (as shown in FIG. 4) for a predefined time interval preferably but not limited to 1 second. Once the user expectorates into the apparatus 100, expectorates 128 falls inside the interior space of the receptacle 102. As soon as the receptacle lid 122 is closed by the user, the ultraviolet (UV) light sources 114 switches on for a predefined time interval preferably but not limited to 1 second to sterilize or disinfect expectorates 128 received or collected in the interior space of the receptacle 102. When the ultraviolet (UV) light sources 114 radiates UV rays (not shown) for the sterilization, the radiated rays strike at the reflective source 106, i.e reflective mirrors configured over the side walls 110 and undergo reflection and this reflection may be repetitive. When the reflected rays fall over the collected expectorates 128 preferably present on the bottom wall 108, the rays disinfect or kill the pathogenic microbial agents like viruses, bacteria, fungi, and other forms of micro organisms that may be present in expectorates. Once expectorates are disinfected or become contaminant free, the disinfected expectorates can be safely disposed without any risk of contamination of environment or infectious diseases originating from them.

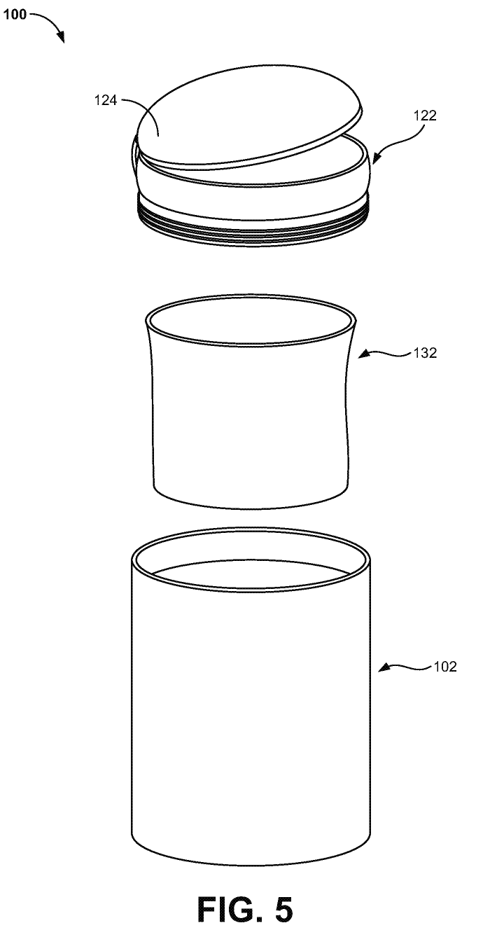

Referring to FIG. 5 shows another alternative exemplary embodiment of the apparatus configured for collection and sterilization of expectorates. As seen, the apparatus 100 will embody similar components such as the receptacle 102, the refractive source 106, one or more ultraviolet (UV) light sources 114, the receptacle lid 122 with the top flap 124, and the power source 112 as described above with reference to FIGS. 1-3. The separator 116 as described above in FIGS. 1-3 may be optionally used. In addition, according to the embodiment, the apparatus 100 may further include an insert 132. The insert 132 can be removably disposed within the interior space of the receptacle 102. The insert 132 may for example be shaped similar to the shape of the receptacle 102. The insert 132 may be transparent and may be made of material that would not hinder the rays radiated from the UV sources 114so that the reflective action of the reflective source 106is properly carried out for disinfection of expectorates collected inside the interior space. The use of removable insert 132 may be preferred for ease in disposing the collected and sterilized expectorates. According to some other embodiment, the removable insert 132 may be made reusable where after each use the removable insert 132 may be sterilized for reuse, according to an exemplary embodiment, the removable and reusable insert 132 may undergo autoclaving for sterilization. However, it should be noted, that any other suitable sterilization process can be adopted for the sterilization of the removable insert 132 to make it fit for next use.

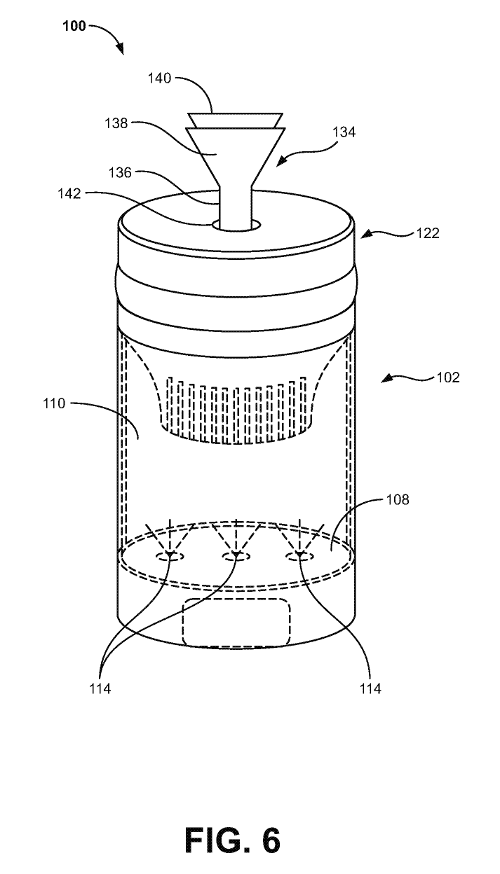

Referring to FIG. 6 shows yet another alternative exemplary embodiment of the apparatus configured for collection and sterilization of expectorates. As seen, the apparatus 100 will embody similar components such the refractive source 106, one or more ultraviolet (UV) light sources 114, the receptacle lid 122, and the power source 112 as described above with reference to FIGS. 1-3. The separator 116 as described above in FIGS. 1-3 may be optionally used. As shown, the receptacle lid 122 may not have the top flap 124 unlike as shown in FIGS. 1-3, instead, the receptacle lid 122 may be configured to have a funnel headed tube assembly 134 as shown in FIG.6.

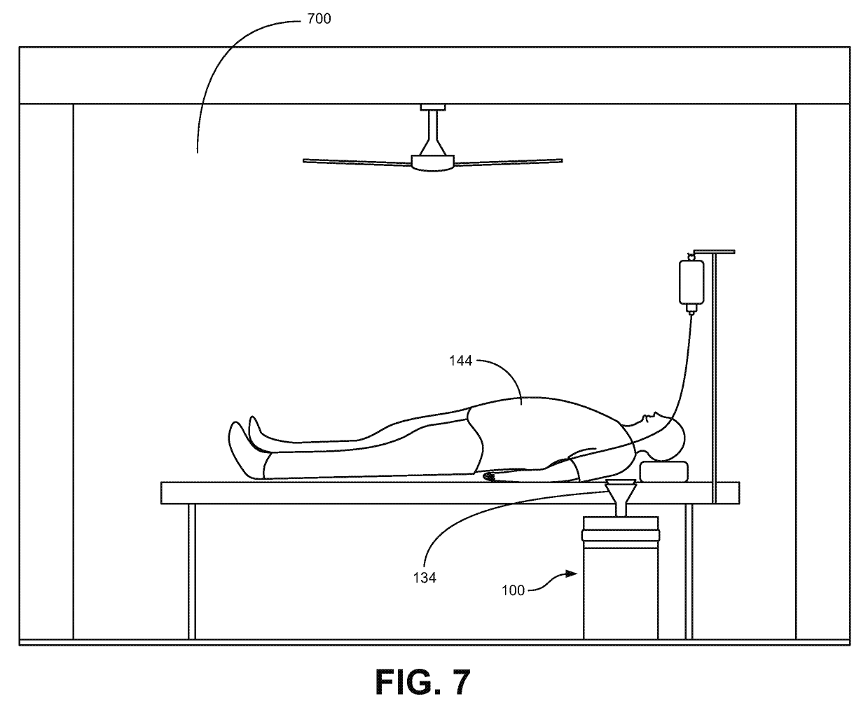

The funnel headed tube assembly 134 may comprise of an elongated flexible tube 136 of a predetermined length including an upper end, and a lower end. The tube 136 may be made of any flexible material preferably of any suitable polymer. The tube 136 may include a cavity with length substantially equal to the predetermined length of the elongated flexible tube 136.The elongated flexible tube 136 may be fixedly or removably attached at its lower end onto a slot 142 disposed centrally on the upper surface of the lid 122. The funnel headed tube assembly 134 may further include a funnel body 138 configured on the upper end of the elongated flexible tube 136 for receiving expectorates. The funnel headed tube assembly 134 further includes a funnel lid 140 for closure of the funnel body 138 when not in use. The funnel body 138 and the funnel lid 140 may be made of any suitable materials known in the art. The funnel tube assembly 134 helps in directing or guiding expectorates into the interior space of the receptacle 102 for collection and sterilization. The embodiment illustrated and shown in FIG. 6 is suitable for use in hospitals or other various health care facilities. As best shown in FIG. 7, the user 144 (specifically a patient) admitted for treatment in a hospital room or hospital environment 700 can have easy access of the apparatus 100 because of the funnel headed tube assembly 134 on the receptacle lid 122. The patient 144 when on bed needs to expectorate, then he can easily grab the tube 136 and pull the assembly 134 near him. Patient 144 just need to remove the funnel lid 140 for expectoration into the funnel body 136. Once the patient expectorates and closes the funnel lid 140, the apparatus 100 functions similar to operation of embodiment described above with reference to FIG. 3. When used in the hospital environment 700, it is the apparatus 100 may be used as a stationary apparatus installable on some specific area say just next to the patient's 144 bed. However, it should be understood that the apparatus 100 may be used in a portable form that can be carried to different locations.

Although not discussed in detail, but the apparatus 100 may be manufactured using any suitable plastic material or any other suitable forms of materials, polymer or metals or any combinations thereof which will have high durability and capable of withstanding the effects of UV rays radiated by the UV sources.

The illustrations of arrangements described herein are intended to provide a general understanding of the structure of various embodiments, and they are not intended to serve as a complete description of all the elements and features of apparatus and systems that might make use of the structures described herein. Many other arrangements will be apparent to those of skill in the art upon reviewing the above description. Other arrangements may be utilized and derived therefrom, such that structural and logical substitutions and changes may be made without departing from the scope of this disclosure. Accordingly, the specification and drawings are to be regarded as illustrative rather than restrictive.

What is claimed is:

1. An apparatus configured for collection and sterilization of expectorates, comprising:
a receptacle including an interior space defined by a bottom wall, and one or more side walls, an opening at a top portion for selectively receiving a separator for expectorates to pass therethrough and be received at said interior space, and a power source;
one or more light sources radiating rays of sufficient wavelengths capable of sterilizing expectorates received at said interior space, wherein said one or more light sources are powered by said power source;
a reflective source surrounding said interior space of said receptacle for substantially reflecting said radiated rays throughout said interior space to effectively sterilize expectorates received at said interior space; and
a receptacle lid removably engaging with said separator to close said opening at said top portion of said receptacle.

2. The apparatus of claim 1, where said one or more light sources is an ultraviolet (UV) light source.

3. The apparatus of claim 1, where said one or more light sources having wavelengths comparable to wavelengths of said ultraviolet (UV) light source.

4. The apparatus of claim 1, wherein said reflective source including a reflective foil.

5. The apparatus of claim 1, wherein said reflective source including one or more reflective mirrors.

6. The apparatus of claim 1, wherein said receptacle lid having a top flap hingedly configured thereon to operate in an open state, and a closed state.

7. The apparatus of claim 6, wherein said top flap when operated in said open state deactivates said one or more light sources.

8. The apparatus of claim 6, wherein said top flap when operated in said closed state activates said one or more light sources.

9. The apparatus of claim 1, wherein said one or more light sources configured onto said bottom wall of said receptacle.

10. The apparatus of claim 1, wherein said one or more light sources configured onto said one or more side walls of said receptacle.

11. The apparatus of claim 1, wherein said one or more light sources configured on an underside face of said receptacle lid, said underside face being opposite to said bottom wall of said receptacle.

12. The apparatus of claim 1, wherein said one or more light sources is configured externally.

13. The apparatus of claim 10, wherein the rays from said externally configured one or more light sources is directed to enter said interior space of said receptacle using a directing mechanism.

14. The apparatus of claim 1, wherein said power source is configured on at least one of: a bottom portion of said receptacle, said one or more side walls of said receptacle, and said receptacle lid.

15. The apparatus of claim 1 further comprising an insert that can be removably disposed within said interior space of said receptacle.

16. The apparatus of claim 1, wherein said separator including a plurality of openings for expectorates to pass and be collected at the interior space.

17. The apparatus of claim 1, wherein said receptacle lid including a funnel headed tube assembly configured thereon.

18. The apparatus of claim 17, wherein said funnel headed tube assembly comprises of
an elongated flexible tube of a predetermined length including an upper end, and a lower end, and including a cavity with length substantially equal to the predetermined length of the elongated flexible tube;
a funnel body configured on said upper end of said elongated flexible tube for receiving expectorates; and
a funnel lid for closure of said funnel body when not in use.

19. The apparatus of claim 17, wherein said funnel headed tube assembly directs expectorates to said interior space of said receptacle for collection and sterilization.
